(19) United States
(12) Patent Application Publication
(10) Pub. No.: US 2026/0202330 A1
(43) Pub. Date: Jul. 16, 2026

(54) REMOTE SUBSTANCE DETECTION SYSTEM, DEVICE AND METHOD

(71) Applicant: Urugus S.A., Montevideo (UY)

(72) Inventors: Nicola Palombo Blascetta, Barcelona (ES); Andres Francisco Brumovsky, CABA (AR); Delfina Rueda, Barcelona (ES); David Ivan Vilaseca, Barcelona (ES)

(21) Appl. No.: 19/136,008

(22) PCT Filed: Dec. 5, 2023
(86) PCT No.: PCT/US2023/082477

§ 371 Date: Jun. 5, 2025

(30) Foreign Application Priority Data
Dec. 5, 2022 (EP) 22383191.8
Apr. 21, 2023 (EP) 23382382.2

(51) Int. Cl. G01N 21/3504; G01N 21/01; G01N 21/31; G01N 21/359

(52) U.S. Cl. CPC G01N 21/3504; G01N 21/01; G01N 21/3103; G01N 21/359; G01N 2021/0106; G01N 2021/3129; G01N 2021/3148

(57) ABSTRACT

A remote substance detection system is provided, the system comprising: at least one light directing element lens; at least one optical multispectral filter; and at least one detector; wherein the optical multispectral filter is configured to have at least one target substance band for detection of at least one target substance and at least one spectral reference band. Also a device and method for remote substance detection is provided.

### CROSS REFERENCE TO RELATED APPLICATION

This patent application claims the benefit of and priority to the European Patent Application No. 22383191.8, filed Dec. 5, 2022, entitled “REMOTE SUBSTANCE DETECTION SYSTEM, DEVICE AND METHOD” and European Patent Application No. 23382382.2, filed Apr. 21, 2023, entitled “REMOTE SUBSTANCE DETECTION SYSTEM, DEVICE AND METHOD” which are hereby incorporated herein in their entirety by reference

### TECHNICAL FIELD

The present invention relates to systems, devices and methods for remote substance detection, such as remote gas detection.

### BACKGROUND

Remote detection of species or substances has many advantages. For example, it may allow to monitor species or substances that have a spectral signature at certain spectral wavelengths or bandwidths, which can be detected with detectors sensitive to those certain spectral wavelengths or bandwidths. However, since different substances have their spectral signatures in different wavelengths, it might be challenging to design remote detection systems that can operate optimally for determining a certain substance.

Examples of species or substances the detection of which is important are gasses such as methane or carbon dioxide, which have an effect in warming the atmosphere and contributing to climate change. Therefore, detection of gasses such as methane or carbon dioxide is of great interest to mitigate the effects of climate change. Different approaches have been developed for methane detection. Typically, spectral measurements are performed from manned or unmanned aerial or space vehicles to estimate whether there is a leak on the ground. Since the sunlight spectrum resembles a 6000 Kelvin black body radiation, it shows a fast intensity decay for wavelengths longer than 2500 nanometers (nm), consequently the most relevant methane absorption bands for sunlight-based techniques are located at 1600 nm and 2300 nm. Furthermore, the atmosphere light transmission is lower at longer wavelengths, hindering light detection from space. At 2300 nm the absorption lines of the same molecule are much stronger, that is, sensitivity is higher in this band than in other bands. This can be seen in FIG. 1, which is a prior art representation from Elena Sánchez-García et. al., titled “Mapping methane plumes at very high spatial resolution with the WorldView-3 satellite” (Atmos. Meas. Tech., 15, 1657-1674, 2022). FIG. 1, as Elena Sánchez-García et. al. describes, shows a comparison of the spectral sampling in shortwave infrared of different spaceborne instruments with potential for methane mapping. All of these instruments sample the methane absorption feature around 2300 nm. The spatial and temporal sampling characteristics of each mission (spatial resolution, temporal resolution, and temporal coverage) are also provided.

For the above reasons, it is common in the art that the 2300 nm band is used for methane detection. However, at 2300 nm, the photon energy and solar radiance are lower, which requires detectors that operate in said band to be quite complex, because they are large, heavy, power consuming, they need cooling, which in some cases can produce large vibrations, and this also translates into high costs.

In aerial or space systems that have limitations of space, power, and/or cost, which for example have small available space, with low energy available, and/or where it is not possible to place elements that vibrate considerably, it is not possible to use detectors as those known in the art that operate in the 2300 nm band.

Substance detection systems and devices in the 1600 nm band, are also known, as seen in FIG. 1. For spectral remote substance detection, multispectral systems or hyperspectral systems can be used. A hyperspectral system comprises many spectral bands, such as tens or hundreds of bands, such as 100 or 200 bands, which provides a continuous spectrum with a certain spectral resolution. A multispectral system is a simpler system which has multiple bands, but a much smaller amount than a hyperspectral system, typically 4 or 5 bands, which provides 4 or 5 numbers per measurement. It thus provides a smaller amount of information than a hyperspectral filter, but it has the advantage that there is also a smaller amount of data generated, and hence processed and downloaded or transmitted from the satellite. Hyperspectral systems are normally based on the use of spectrometers, devices that are typically too voluminous and heavy for efficient integration into micro/nanosatellites. The use of linear variable filters constitutes a valuable alternative for the design of hyperspectral payloads, but at the state-of-the-art, there are no such devices capable of providing a full-width half-maximum (FWHM)—which gives information about the filter bandwidth or spectral resolution—finer than 15-20 nm at desired wavelengths, strongly limiting the final spectral resolution of the system. On the other hand, it is possible to fabricate custom multispectral systems with a limited amount of bands and an FWHM of 10 nm or less. Known systems in the art are multispectral and work in the 1600 nm band. However, they present several problems.

Typically, multispectral sensors are of general purpose, hence the bands are uniformly distributed in the spectrum, and additionally they do not provide for albedo correction. The albedo refers to the reflectance of the surface of the earth, in other words, the albedo is the measure of the diffuse reflection of solar radiation out of the total solar radiation, and it can be measured on a scale from 0, corresponding to a black body that absorbs all incident radiation, to 1, corresponding to a body that reflects all incident radiation. It was found to be interesting to determine how deep the absorption lines of FIG. 1 are. This can be done very well with a hyperspectral system, however it is very uncommon to do it in a multispectral system. What is commonly done is to work in the 2300 nm band, as seen in FIG. 1, for example the lines corresponding to Worldview-3 (the horizontal lines 102 at the same height named B1-B8), which have had very good results in methane detection. However, it can also be seen that Worldview-3, Sentinel-2 (horizontal lines 104 at the same height named B11 and B12) and Landsat-8 (horizontal lines 106 at the same height named B6 and B7) also work in the 1600 nm area with multispectral systems, with defined bands. However, they typically do not perform a true albedo correction. They instead perform a ratio between two bands, for example, band B7 divided by band B5 of the Worldview-3 line. This is because they see that band B7 seems to have more methane absorption than B5, and they divide B7 by B5 and decide that the result will be the transmittance of the plume. This is because, if there is a leak of methane in the ground, light can pass through the plume that is left by the leak. If there is a leak there, there is more methane absorption, and therefore one would have to see more absorption in the spectrum absorption lines. However, a problem with this approach is that the band used as a control band (B5 in this case) can also partially absorb methane, thus not being a perfect control. An additional and perhaps more important problem of this approach is that it does not take into account the albedo variations which might affect the two bands (B5 and B7) in a different way, hence their ratio would not only be the result of a methane absorption variation, but also of the albedo variation. Therefore, this ratio would not be able to distinguish between a variation in methane concentration or albedo.

In addition, another problem is that what is done in the art as criterion for selecting the bands is that they have to be sufficiently close and correlated in the spectrum, as seen for example in bands B5, B6, B7, B8 (102) of Worldview-3 in FIG. 1, which are close to each other. However, they are not pure bands, in the sense that in each band, which has a width of around 40 nm-50 nm, there is not only methane absorption, but also absorption of other gasses, such as carbon dioxide (CO₂), water (H₂O), and/or other gasses. As a result, the results may not be as accurate as desirable.

These are examples of the challenges faced when designing systems for remote substance detection. It would therefore be desirable to have aerial or space systems, devices and methods for species, material or substance detection, which are less complex and have a lower manufacturing and operating cost than the detectors known in the art, while maintaining or improving the accuracy in species, material or substance detection, and a method of designing a filter that accurately detects target substances and that can be used in said aerial or space systems, devices and methods for species, material or substance detection.

### SUMMARY

Considerable advantages can be achieved with embodiments of the present disclosure, which provides systems and devices for species, material or substance detection, such as for methane detection, and methods of designing, manufacturing and operating the same, that can maintain or improve the accuracy in substance detection, maximizing signal to noise ratio (SNR) in said substance detection, while being relatively simple and inexpensive to manufacture and operate. The systems, devices and methods of the present disclosure are designed to select the amount, position and width of spectral bands that will allow for an accurate detection of the desired substance(s), maximizing the SNR of the measured signal. The systems, devices and methods of the present disclosure are thus suitable for small aerial or space vehicles, such as small satellites or secondary payloads of satellites. In some embodiments of the present disclosure, systems and devices for substance detection operate in the 1600 nm band which, as seen in FIG. 1, also has relatively strong methane absorption lines, although less strong than in the 2300 band, but puts less strong requirements on the complexity and size of the detectors used.

Systems, devices and method of the present disclosure have advantages which include accurate, simpler, smaller, more compact, cheaper detection systems and devices that have a much higher scalability than known large complex systems, and that can be integrated faster and in a simpler manner in aerial systems or spacecrafts such as satellites, thus also allowing for an increase in the coverage with time, and an increase in the revisit time.

The present disclosure provides a remote substance detection system, the system comprising: at least one light directing element; at least one optical multispectral filter; and at least one detector; wherein the optical multispectral filter is configured to have at least one spectral reference band and at least one target substance band for detection of at least one target substance (in other words, at least one target substance band for detection of at least one target substance and at least one spectral reference band).

According to embodiments of the present disclosure, the at least one spectral reference band and the at least one target substance band are determined based at least in part on at least one of: a spectral proximity between bands (for example a spectral proximity between the at least one spectral reference band and the at least one target substance band or a spectral proximity between several target reference bands), a correlation between bands (for example a correlation between the at least one spectral reference band and the at least one target substance band or a correlation between several target reference bands), a maximum total absorbance of at least one substance present in a spectral area of interest, a minimum total absorbance of at least one substance present in the spectral area of interest, a maximum relative absorbance of at least one substance present in the spectral area of interest, a minimum relative absorbance of at least one substance present in the spectral area of interest, a maximum between the total and relative absorbance, or a minimum product between the total and relative absorbance.

According to embodiments of the present disclosure, the at least one target substance and/or the at least one substance is at least one of oxygen, nitrogen, methane, water, carbon dioxide, carbon monoxide or nitrous oxide.

According to embodiments of the present disclosure, the at least one spectral reference band is selected to be located in spectral areas where an absorbance of the at least one target substance is lower than an absorbance threshold or lower than a pure band threshold, and/or in spectral areas where a relative absorbance of the at least one target substance with respect to other substance or substances is low enough that a change in absorption of the other substance or substances does not depend on a change in the at least one target substance. According to embodiments of the present disclosure, an amount, position in the spectrum and/or bandwidth of the at least one spectral reference band is based at least in part on a number of target substance bands, a spectral distance between the target substance bands, and a frequency of variation of albedo in a spectral range of interest.

According to embodiments of the present disclosure, the optical multispectral filter is configured to have at least two spectral reference bands. According to embodiments of the present disclosure, an interpolation curve is obtained using measurements at the at least two spectral reference bands as interpolation points, in order to obtain a relative variation.

According to embodiments of the present disclosure, the measurements correspond to values of a radiance on top of atmosphere (TOA).

According to embodiments of the present disclosure, the system further comprises a coating layer.

According to embodiments of the present disclosure, the at least one multispectral optical filter comprises one first target substance band for methane detection, preferably centered at around 1647 nm. According to embodiments of the present disclosure, the at least one multispectral optical filter further comprises one second target substance band for methane detection, preferably centered at around 1670 nm. According to embodiments of the present disclosure, the at least one multispectral optical filter further comprises one third target substance band for carbon dioxide detection, preferably centered at around 1600 nm.

According to embodiments of the present disclosure, the at least one multispectral optical filter comprises one first target substance band for carbon dioxide detection, and preferably one second target substance band for methane detection and one third target substance band for methane detection, wherein preferably the first target substance band for carbon dioxide detection is centered at around 1600 nm, the second target substance band for methane detection is centered at around 1647 nm and the third target substance band for methane detection is centered at around 1670 nm.

According to embodiments of the present disclosure, the at least one multispectral optical filter comprises one carbon dioxide band for carbon dioxide detection centered at around 1600 nanometers, one first methane band for methane detection centered at around 1647 nanometers, and one second methane band for methane detection centered at around 1670 nanometers. According to embodiments of the present disclosure, a first reference band is centered at around 1620 nanometers and a second reference band is centered at around 1695 nanometers.

The present disclosure provides an optical filter comprising: one or more filter sections, wherein at least one of the one or more filter sections is a target substance filter section for detection of at least one target substance and at least another one of the one or more filter sections is a spectral reference filter section.

According to embodiments of the present disclosure, the at least one spectral reference filter section and the at least one target substance filter section are determined based at least in part on at least one of: a spectral proximity between at least one spectral reference band and at least one target substance band, a spectral proximity between several target reference bands, a correlation between the at least one spectral reference band and the at least one target substance band, a correlation between several target reference bands, a maximum total absorbance of at least one substance present in a spectral area of interest, a minimum total absorbance of at least one substance present in the spectral area of interest, a maximum relative absorbance of at least one substance present in the spectral area of interest, a minimum relative absorbance of at least one substance present in the spectral area of interest, a maximum between the total and relative absorbance, or a minimum product between the total and relative absorbance.

According to embodiments of the present disclosure, the optical filter is configured to be disposed in an optical path of a lens and focal plane array, and is configured to filter light that passes through the lens to reach a planar surface of the focal plane array.

According to embodiments of the present disclosure, the target substance filter section is a first target substance band for methane detection, preferably centered at around 1647 nm. According to embodiments, the target substance filter section is a second target substance band for methane detection, preferably centered at around 1670 nm. According to embodiments, the target substance filter section is a third target substance band for carbon dioxide detection, preferably centered at around 1600 nm.

According to embodiments of the present disclosure, the one or more filter sections comprise one first target substance band for carbon dioxide detection, and preferably one second target substance band for methane detection and one third target substance band for methane detection, wherein preferably the first target substance band for carbon dioxide detection is centered at around 1600 nm, the second target substance band for methane detection is centered at around 1647 nm and the third target substance band for methane detection is centered at around 1670 nm. According to embodiments of the present disclosure, at least one spectral reference filter section is centered at around 1620 nanometers and at least one spectral reference filter section is centered at around 1695 nanometers.

According to embodiments of the present disclosure, the one or more filter sections are arranged as discrete filter bands, filter arrays or mosaics, or any combination of these.

The present disclosure provides a computer-implemented method of remote substance detection, comprising the steps of: determining at least one spectral reference band for an optical multispectral filter to be used in a remote substance detection system; determining at least one target substance band for the optical multispectral system; and designing the optical multispectral filter based at least in part on the at least one spectral reference band and the at least one target substance band.

The present disclosure provides a computer-implemented method of remote substance detection, comprising the steps of: determining at least one target substance band for an optical multispectral filter to be used in a remote substance detection system; determining at least one spectral reference band for the optical multispectral filter; and designing the optical multispectral filter based at least in part on the at least one target substance band and the at least one spectral reference band.

According to embodiments of the present disclosure, determining the at least one target substance band is based at least in part on at least one of: a spectral proximity between the at least one spectral reference band and the at least one target substance band, a spectral proximity between several target reference bands, a correlation between the at least one spectral reference band and the at least one target substance band, a correlation between several target reference bands, a maximum total absorbance of at least one substance present in a spectral area of interest, a minimum total absorbance of at least one substance present in the spectral area of interest, a maximum relative absorbance of at least one substance present in the spectral area of interest, a minimum relative absorbance of at least one substance present in the spectral area of interest, a maximum between the total and relative absorbance, or a minimum product between the total and relative absorbance.

According to embodiments of the present disclosure, determining at least one spectral reference band comprises determining at least two spectral reference bands. According to embodiments of the present disclosure, determining the at least two spectral reference bands comprises selecting the at least two spectral reference bands to be located in spectral areas where an absorbance of a target substance is lower than an absorbance threshold and/or in spectral areas where a relative absorbance of the at least one target substance with respect to other substance or substances is low enough that a change in absorption of the other substance or substances does not depend on a change in the at least one target substance, and/or wherein preferably wherein the method preferably comprises determining a number and position of the spectral reference bands based on a number of target substance bands, a spectral distance between the target substance bands, and a frequency of variation of albedo in a desired spectral range.

According to embodiments of the present disclosure, the method further comprises obtaining an interpolation curve using measurements at the at least two spectral reference bands as interpolation points, wherein preferably the measurements correspond to values of a radiance on top of atmosphere.

The present disclosure provides a computer-implemented method for data analysis, based at least in part on an architecture of the system for remote substance detection as described in the present disclosure, to maximize signal to noise ratio, SNR, for a target substance detection, the analysis comprising: obtaining a multispectral image; analyzing a first spectral band of the multispectral image, wherein the first spectral band is configured to detect at least one target substance; analyzing at least a second spectral band and a third spectral band, wherein at least the second spectral band and the third spectral band are configured to detect at least a substance different than the target substance; and determining information related to the at least one target substance based at least in part on the analysis of the first spectral band and on the analysis of the second spectral and the third spectral band.

Further advantages can be achieved with embodiments of the present invention, by using multispectral systems and devices with specifically designed spectral bands, and with albedo correction, that optimize the accuracy of substance, such as gas (e.g methane) detection. Because of the smaller amount of information generated, and the thinner spectral resolution, some embodiments of the present disclosure use multispectral systems and devices for methane detection. It should however be noted that hyperspectral systems in the 1600 nm band, are also suitable alternatives encompassed in the present disclosure.

Further features and advantages, as well as the structure and operation of various embodiments are described in detail below, with reference to the accompanying drawings. It is noted that the invention is not limited to the specific embodiments described herein. Such embodiments are presented herein for illustrative purposes only. Additional embodiments will be apparent to persons skilled in the relevant art(s), based on the teachings contained herein.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items.

FIG. 1 shows a prior art graph from Elena Sánchez-García et. al. showing a comparison of the spectral sampling in shortwave infrared of different spaceborne instruments with potential for methane mapping.

FIG. 2 illustrates the absorption spectra of different substances.

FIG. 3 schematically shows a representation of how substances are detected in some embodiments of the present disclosure.

FIG. 4 is a representation of absorption lines of different gasses in different spectral bands.

FIG. 5 is a representation showing absorption bands of carbon dioxide.

FIGS. 6 (a-e) are graphs showing criteria for selection of bands of carbon dioxide.

FIG. 7 is a representation showing absorption bands of methane.

FIGS. 8 (a-e) are graphs showing criteria for selection of bands of methane.

FIG. 9 is a representation showing absorption bands of methane.

FIGS. 10 (a-e) are graphs showing criteria for selection of bands of methane.

FIG. 11 is a graph showing a graphical representation of a suitable reference band or albedo band.

FIG. 12 is a representation showing the absorbance as a function of wavelength for (A) H2O and (B) CH4.

FIG. 13 illustrates the Radiance on top of atmosphere (TOA) between 1600 nm to 1800 nm.

FIG. 14 illustrates a side view of an example substance detection device, according to embodiments of the present disclosure.

FIG. 15 illustrates an example of a detector and a multispectral optical filter, according to embodiments of the present disclosure.

FIG. 16 shows a method to design a multispectral filter according to some embodiments of the present disclosure.

FIG. 17 shows a method for data analysis according to embodiments of the present disclosure.

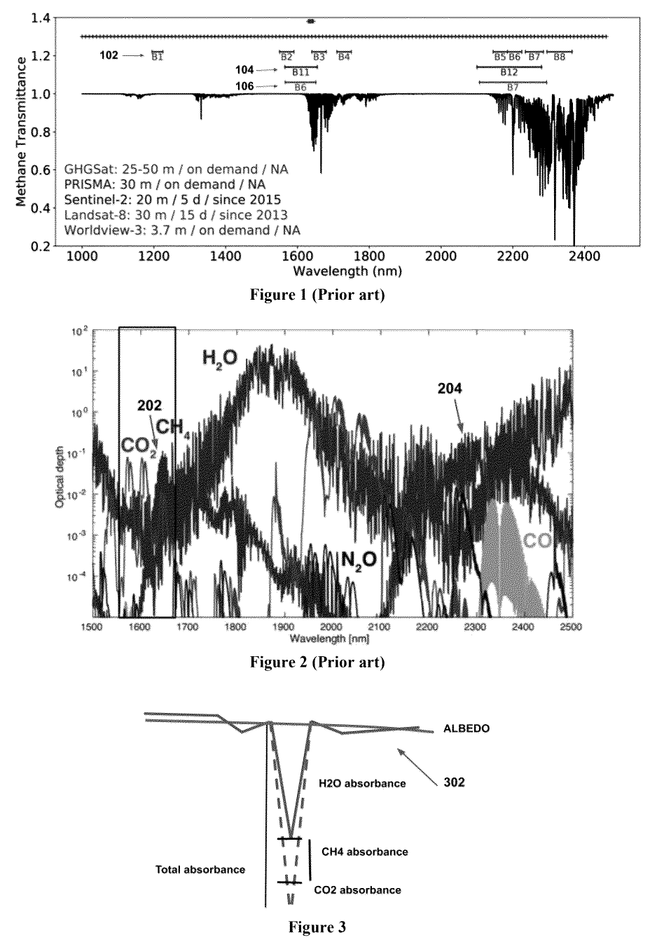

### DETAILED DESCRIPTION OF EMBODIMENTS

Embodiments include a species, substance or material detection system, comprising at least one light directing element (or light collecting element, or light focusing element), an optical filter, and a sensor array (e.g., an array of pixel sensors). The substance detection system may be on board a vehicle such as an aerial or satellite vehicle. The substance detection system may be part of a moveable platform, such as an aerial or satellite system. The substance detection system may likewise be part of a ground-based platform. In some instances, the moveable or ground-based platform is not designed to move during substance detection, while in other implementations, the moveable or ground-based platform is in motion during substance detection. The substance detection system according to embodiments may include imaging spectrometers, variable filters, or other mechanisms that cause the pixel sensors within portions of the sensor array (also referred to as detector or focal plane array) to be associated with a narrow band of wavelengths. The light directing element may be a partially or completely transmissive optical element which directs a light beam by means of refraction, such as a simple lens, a compound lens including several simple lenses, or a mirror, a telescope or any element configured to direct electromagnetic radiation. The light directing element may be configured to direct, that is, collect and focus, light so that it reaches other parts of the system.

Embodiments provide specifically engineered filters (or just filter) for species, substance or material detection, for example for gas detection e.g. CH₄ and/or CO₂ detection. The filter may be an optical filter, including, but not limited to a multispectral filter, low-pass filter, a high-pass filter, a band-pass filter, or any combination of these. For example, an optical bandpass filter may contain one or more regions of the filter configured to selectively transmit a portion of the electromagnetic spectrum while attenuating or reflecting other wavelengths. The one or more regions may be linear, that is, a linear bandpass filter may have discrete regions of the filter that allow a high transmission across narrow bandwidths while attenuating unwanted light to maximize image capture at the required wavelength. Other examples of optical filters may be used with embodiments described herein and are contemplated herein as providing the features and benefits described. For instance, a notch filter, a filter that attenuates a narrow band of wavelengths, may be used with the systems and methods described herein. Similarly, an optical bandpass filter, one that allows a band of wavelengths to pass, may likewise be used. One or more filters may be used to selectively attenuate or pass desired wavelengths in order to detect different target substances, detectable within one or more spectra of interest (bands).

In some embodiments, the optical filter of the substance detection system is a multispectral optical filter. As used herein, a multispectral optical filter, refers to an optical filter that allows various wavelengths of light to pass through portions thereof. The multispectral optical filter may comprise at least one reference band, and at least one target substance band. In some embodiments, the multispectral optical filter may comprise at least two reference bands, and at least one target substance band.

In some instances, the amount, position in the spectrum, and/or bandwidths of the target bands and the reference bands are selected based, at least in part, on the absorbance curves of the target substance(s) and other substances detectable in the same area(s) of the spectrum. For example, the amount, position in the spectrum, and/or bandwidths of the target bands and the reference bands may be selected based, at least in part, on the absorbance curves of water and the target gasses (i.e. the gasses to be detected). In some instances, the amount, position in the spectrum and/or bandwidths of the reference bands may in addition or alternatively be selected based at least in part on the number target bands of the target substance(s), the spectral distance between said target bands, and the frequency of variation of the albedo, that is, how fast the variations in albedo are, in the spectral range of interest.

In the last decades the general concern for the problem of global warming increased considerably due to its catastrophic impact on the climate, hence on the entire biosphere of planet Earth, as discussed in “The Closing Window”, Emissions Gap Report 2022, United Nations Environment Programme, 2022, and “2022 State of Climate Services”, 1301, World Meteorological Organization, 2022. Methane (CH₄) is a major greenhouse gas second only to carbon dioxide (CO₂), and in the first 20 years after emission it is 80 times more powerful than CO₂ at trapping heat in the atmosphere, as discussed in “Control methane to slow global warming fast”, Nature, 596, 2021, and “Global Methane Assessment”, United Nations Environment Programme, 2021. On the other hand, methane breaks down much more quickly than CO₂, thus methane emissions reduction provides a valuable actionable tool for global warming mitigation in the short time scale.

Methane emissions have been detected along gas pipelines, offshore extraction plants, coal mines, cow farming sites and landfills. To act on methane emissions requires the ability to detect, measure, quantify and monitor in time such sources, in order to provide decision makers and legislators with precise and reliable data. Furthermore, the nature of the problem requires big mapping capacity (entire planet Earth), with high frequency of remapping and high enough spatial resolution to provide actionable data, all with a sustainable and cost effective approach.

Satellite technology has been proven to be one of the most promising for atmospheric methane detection and monitoring. Nevertheless, the requirement for frequent remapping of big areas puts relatively small satellites like micro/nanosatellites (or in general, satellites of less than 500 kg) in a better position than big traditional ones due to their higher scalability for fast constellation realization.

In the field of spectroscopic methane detection from space with sunlight illumination, two main absorption bands are targeted at 1.6 and 2.3 μm wavelengths. Even though the methane molecular absorption strength is higher at 2.3 μm, the detection of light at this wavelength requires the use of low temperature detectors (150 K), which at the state-of-the-art are relatively big, heavy, with high energy consumption and introduce vibrations that hinder their integration into microsatellites/small cubesats. On the other hand, the detection band at 1.6 μm which shows slightly lower sensitivity to methane absorption, is technologically compatible with integration in small and rapidly scalable satellites.

From the payload architecture point of view, two main technologies are considered: hyperspectral and multispectral. Hyperspectral systems provide a complete set of spectral data, but they require spectrometers which are too big and heavy to be integrated in small cubesats (≤2U). Additionally, the download from space of big amounts of data is limited by the well-known problem of limited download bandwidth.

On the other hand, multispectral systems are based on relatively simpler architectures as optical filters and can easily be integrated in both microsatellites as secondary payloads or small cubesats (≤2U). Moreover, they produce 1-2 orders of magnitude lower amounts of data, providing for a much smaller data set to download down to Earth or to transmit to other devices or systems.

Unfortunately, the performance in terms of methane spectroscopic detection from space of multispectral systems is poorer than hyperspectral systems, especially in the case of heterogeneous surfaces on Earth (heterogeneous surfaces may comprise surfaces where the landscape changes, for example from desert, to water, to forest, or the like, as opposed to homogeneous surfaces which would show less variation, for example surfaces including only desert). This happens because the gas concentration retrieval is based on the measurement of photons reaching the detector at the methane absorption bands energies, and such number of photons depends also on the other atmospheric gasses absorptions and the surface reflectance (albedo). Hence, an efficient methane concentration detection relies on a good enough bands spectral selectivity and albedo retrieval to disentangle the methane absorption from such other effects. Additionally, among other atmospheric gasses, it is particularly important to be able to measure and disentangle water concentration, especially in off-shore measurements.

The solutions according to some embodiments of the present disclosure include a primarily CH₄ and optionally, a secondarily CO₂ detection payload (or a primarily CO₂ detection payload, and optionally a secondarily CH₄ detection payload), both based on an original, optically simple, compact, light and low energy consumption architecture with a custom multispectral optical filter working in the spectral range 1.6-1.7 μm integrated with a commercial lens and a standard InGaAs detector.

The filter according to some embodiments is constituted by 5 bands of 10 nm FWHM each, best trade-off between good enough spectral selectivity and sensitivity and robust SNR with the typical photon budget available for a micro/nanosatellite. Photon budget refers to the number of detectable photons, or the amount of available photons.

The filter according to some embodiments has one band for the CO₂ detection centered at 1600 nm wavelength and two bands for CH₄ detection centered at around 1647 and around 1670 nm wavelengths. The bands have been chosen to maximize absorption and selectivity (purity) and minimize the other atmospheric gasses absorptions in the SWIR range 1600-1700 nm. Two bands have been spectrally allocated to provide albedo retrieval and humidity level measurement. The first band has been located at around 1620 nm in a spectrally pure region, where there is absence of absorption from all atmospheric gasses. The second one has been located at around 1695 nm where the absorption is dominated by water (>98% with a humidity of 2.5%). Without loss of generality, the methods, systems and devices of embodiments of the present disclosure may be implemented for any gas and for any number of spectral bands.

An original ad hoc data analysis approach has been developed based on an albedo retrieval method, which makes use of an interpolation method, such as a linear interpolation, between the two albedo bands, spectrally closely correlated to the CH₄ and CO₂ bands to retrieve the albedo. Additionally, the ratio between the two albedo bands may provide a measurement of additional parameters, such as humidity, a critical aspect in offshore CH₄ detection. This can be done by choosing the two albedo bands in such a way that one of them absorbs H₂0, and the other one is mostly insensitive to any substance absorption, in other words, does not absorb any substance (above a pure band threshold). The ratio of both bands thus allows to determine the presence of water, and hence to measure humidity. A substance different from water could also be measured if the albedo band was sensitive to another substance, which was also different from CH₄.

Finally, the payload concept according to some embodiments is compatible at least with a small cubesat of <2U (double unit) size or with a typical microsatellite secondary payload integration.

In some embodiments of the present disclosure, multispectral substance detection systems are used. In some embodiments, in which for example substances such as CH₄ and/or CO₂ are the target substances, the multispectral substance detection systems are configured to operate at around the 1600 nm spectral band. Because the multispectral system needs to have discrete bands, it needs to be decided how many bands there need to be, where these bands are to be placed, and which width these bands are to have. FIG. 2 is a representation provided in document from Daniel J. Jacob et. al., titled “Satellite observations of atmospheric methane and their value for quantifying methane emissions” (Atmos. Chem. Phys., 16, 14371-14396, 2016). FIG. 2 shows the absorption spectra between 1500 nm and 2500 nm, for different substances, and it can be seen that the absorption spectrum of CH₄ has two maxima, one 202 at around 1600 nm and one 204 at around 2300 nm. It needs to be decided where to place the target bands for detection of the multispectral system, which criterium to use, and where to place the reference bands, that is, how to extract the albedo.

Typically, with a hyperspectral system one can measure the whole spectrum, where there is a low frequency, which is a way of seeing how the spectrum moves or changes in general terms, and then there are the thin absorption lines. However, to detect thin absorption lines with hyperspectral systems, enough light is required. This is however not suitable for small satellites or payloads, which may not have enough light, due to, among other reasons, the sizes and types of detectors that they comprise, and may have low spectral resolution, of about 10 nm, which does not allow to measure single absorption lines. Therefore, a solution proposed in embodiments of the present disclosure to perform accurate substance detection comprises detecting a general tendency of the spectrum, the lowest frequency of variation, in a similar way as if it was an electronic signal, by defining at least two reference bands (albedo bands) and performing an interpolation function between the measurements performed in said reference bands to determine the general tendency variation of the spectrum. The solution proposed in embodiments of the present invention then detects the local minima, which are caused by the target substance(s), whose presence and amount is to be detected, and by any other substances present. The target substance(s) and the other substance(s) may include at least one of CH₄, CO₂, but also water, and any other suitable substance that may be present, and therefore certain criteria have been chosen to determine the amount, position and width of the bands. This can be seen schematically in FIG. 3, representing the general tendency of the spectrum as the albedo 302. Said criteria to determine the amount, position and width of the bands (the target band(s) and/or the reference band(s)) include not only spectral proximity and correlation of the bands, as commonly done in the art, but it also includes the number of bands, the frequency of change of the albedo in the desired area of the spectrum, and also in some cases it includes maximizing and/or minimizing the total and/or relative absorbance of each substance (methane, water, carbon dioxide, etc.), maximizing and/or minimizing the product between the total and relative absorbance, which provides a more absolute value, or other suitable criteria.

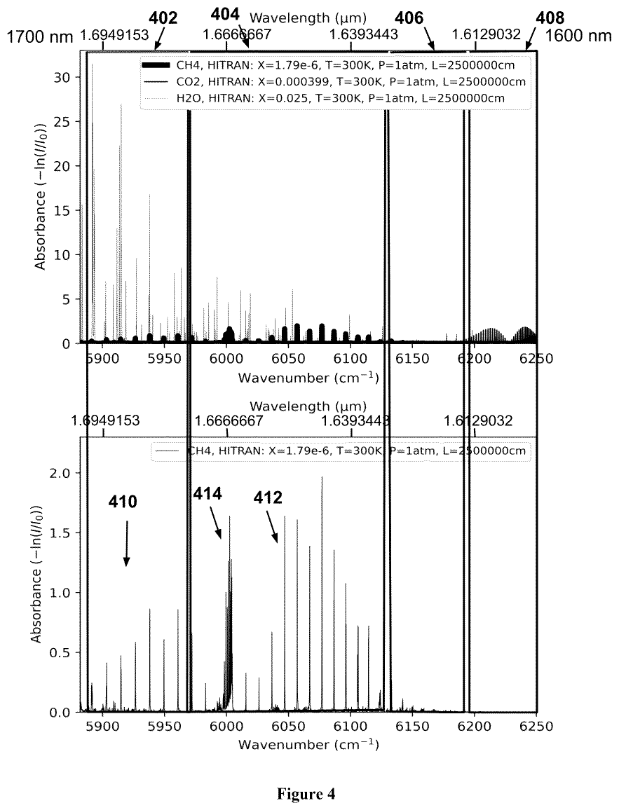

Calculations according to at least some embodiments can be done based on spectroscopy, so as to allow to see the absorption lines of each substance of interest. These calculations may be used to determine the number and position of the bands. A representation of the results of an example of such calculations, performed with a calculation system based on spectroscopy (using the HITRAN database and the tool SpectralPlot), can be seen in FIG. 4. The graphs show the range between 1600 nm and 1700 nm. The upper side of the graph represents the absorption lines of water, carbon dioxide and methane. However, the absorption lines of the water are dominant, therefore, the lower side of the graph represents the same values but the absorption lines of the water have been removed, so as to better visualize the absorption lines of the methane and carbon dioxide. In this graph of the lower side, the absorption bands of the methane can more clearly be seen. It can be seen that there are two comb-like structures 410 and 412 separated by an intense and thin band 414 in the middle. A suitable place for the target bands for detecting methane may be where there is a lot of methane absorption (where the peaks 412 and 414 are in the graph of the lower side).

For detecting the albedo, it may be suitable to place the reference bands in areas where the target substance(s), such as methane, do(es) (almost) not absorb, because this allows to have an upper baseline. If the target bands are placed where there is a lot of methane absorption (where the peaks 412 and 414 are in the graph), in those places it may be possible to see the emission minima, depending on the gas absorption. In other words, embodiments of the present invention determine the position and width of the bands for detecting albedo in the areas where there are no peaks in the absorption of the target substance that is being detected, such as methane. The idea is not only searching for a band close to where there is a maximum of the target substance, but in particular and differently to what is commonly known, is to have at least two albedo bands, not just one, and at least one of the albedo bands (also known as reference bands), but preferably the two albedo bands, are to be located in areas which are spectrally clean, that is, where the target substance and preferably other substances absorb below a certain threshold, a pure band threshold. For example, one albedo band can be located at the rectangle 406 of FIG. 4, which is an area with very little gas absorption. The rectangle 404 has a lot of methane absorption, and therefore may not be suitable for albedo detection. The rectangle 402 also has methane absorption, but as can be seen in the upper side graph, there is a very high level of water absorption as well. Therefore, the inventors realized that, instead of using only one band of albedo, using two bands may be advantageous, and band 402 can be used as the second albedo band.

As can be seen in the rectangle 406 to the right, it corresponds to a pure band because there is (almost) no absorption of any gas, or no absorption of any gas above a pure band threshold. Therefore, when measuring light in said area, the baseline is very high, in view that nothing absorbs. However, on the other band, corresponding to rectangle 402 to the left, there is methane absorption but also a lot of water absorption. It is thus not a pure band, but the relative absorption of methane is very low. It is a band where there is absorbance but almost entirely of gasses that are not methane, that is, there is absorbance of substances that are not the target substance, but there is no absorbance of the target substance(s) or the absorbance is below an absorbance threshold, or the relative absorbance of the target substance(s) with respect to the substance(s) other than the target substance is so low that a change in the absorption of the substance(s) other than the target substance cannot be correlated with (does not depend on) a change in the target substance. In the case of the target substance being methane, if it is assumed that in case of a localized methane leak the humidity does not change abruptly, it can be assumed that the variation in transmission in this band 402 is also mostly due to albedo variations. In the worst case, even if the transmission changes because of strong humidity variations, it will surely not depend on methane absorption, and therefore it can be used as a reference band for methane detection. Thus, said band 402 can be a suitable reference band, or albedo band, and it can be used as a criterion to have the upper reference, in cases where methane is the target substance. Lastly, more to the right, rectangle 408 shows two bands with absorbance of carbon dioxide higher than an absorbance threshold, so these bands can also be defined as suitable bands to detect carbon dioxide.

Another important factor to take into account when selecting the bands, apart from the location of each band, is the width of each band. Typically, in this type of measurements, the thinner the bands, the better the spectral resolution. However, there is also a tradeoff to consider, because very thin bands are costly (the filters required are much more expensive, because to get such transmission lines it is required to deposit many more layers of material, so the complexity of design and mostly fabrication increases considerably), and allow to collect slight amounts of light (because optical custom transmission filters with very thin transmission lines, due to the fact that require many layers, have in general a relatively low transmission even in the transmission bands, having a detrimental impact on the available photon budget). Generally, in substance detection systems such as micro or nanosatellites, and/or secondary payloads, the amount of photons that can be collected is very small, and it is therefore not possible to have very thin bands. The bands of the Worldview-3 satellite, for example, have a width of 40 nm-50 nm. This width allows to collect a lot of photons, but also, if the band is too broad, it becomes difficult to have spectral selectivity. As can be seen in FIG. 4, the rectangle 408, for example, has a width of 10 nm, the rectangle 404 has a width of 50 nm, and the rectangle 406 has a width of 10 nm. In order to have a band where there is suitable absorption, to be used as a target band or as a reference band, preferably it should be wide enough to collect enough photons to measure the signal, but thin enough to provide enough spectral selectivity. For example, a band with suitable absorption can have a width of 10 nm, and may give a good compromise between spectral selectivity and amount of collected light. However, a band that has 20 nm of width may increase the probability of collecting methane from one side (from the 404 band) and carbon dioxide from the other side (from the 408 band). For this reason, a reference band 10 nm wide is suitable, however it should be understood that also other band widths may be suitable, since this may be applicable for a certain target substance and a certain spectral region, but not for others. For example, depending on the target substance, the bandwidth of the spectral area in which the target substance absorption lines are located may vary. For example, at around 2300 nm the methane absorption lines are much more dispersed than at 1600 nm. What is important in order to decide the bandwidth is to have a tradeoff that allows to collect enough photons but that also provides enough spectral selectivity, but depending on the target substance and the spectral range where it is to be measured, since this is very specific of the target substance that is to be detected, and also of the spectral range used. For example, the 2300 nm band for detecting methane is much more continuous, less discrete, where, in order to have an albedo band, it may be suitable to have a band that is thinner than for the 1600 nm band, such as of 5 nm of spectral resolution (FWHM). The bandwidth and bands location may be selected based at least in part on the target substance absorbance (or transmittance), the characteristics of the detector such as the sensitivity, the spectral range used for detection, or any combination of these.

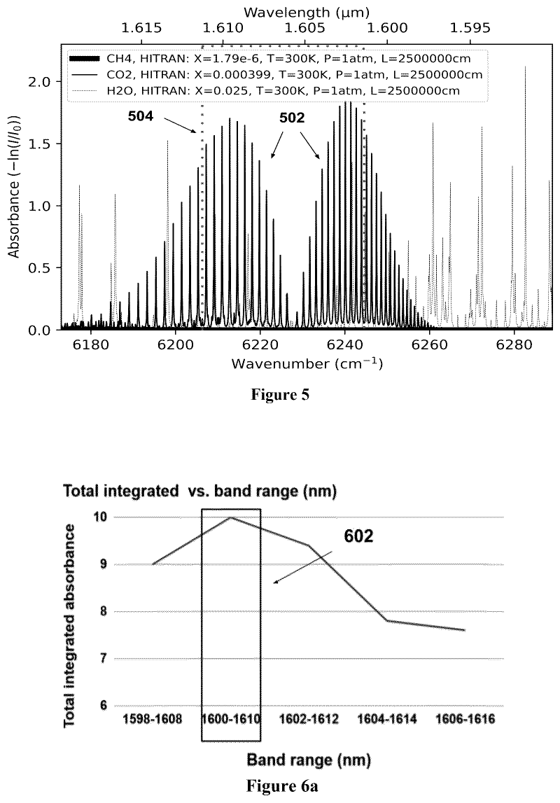

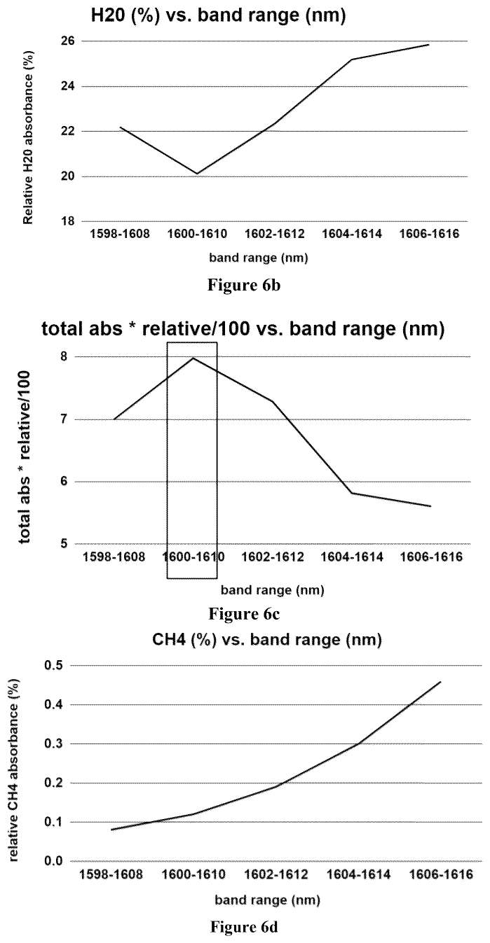

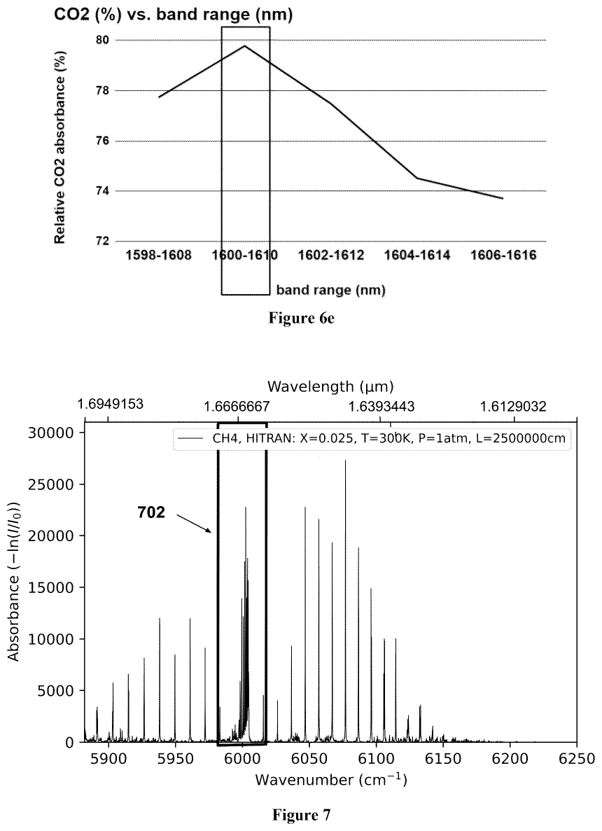

FIG. 5 shows the absorption bands of carbon dioxide. The lines 502 represent the carbon dioxide absorption lines. The dashed line 504 delimits a window of a 10 nm-width band. A suitable position for this 10 nm-width band is to be found. FIG. 6 shows examples of the criteria used to choose the position of the band(s) to detect presence of carbon dioxide, according to embodiments of the present disclosure. A first step may include defining a suitable width for placing the band(s), and a second step may include finding a suitable position for the band(s), or vice versa, that is, first determining the position and then determining the width. As an alternative, there might be a first step of defining a broader suitable spectral band (i.e. a band range), by looking at/analyzing the absorption lines of the target substance and other substances which might contribute but are to be excluded. With this, it can be decided what is the rough spectral position of the band and the maximum FWHM that can be afforded keeping good spectral selectivity. Once this rough spectral position is decided, there can be a second step and third step of (in any order) defining a suitable width and a suitable center position for the band within the band range, i.e. the broader suitable spectral band. FIG. 6a shows the absorbance of all gasses present in the band range 1598 nm-1616 nm with respect to different placements of the 10 nm-width band. This band range between 1598 nm and 1616 nm was chosen based at least in part on the determination, as shown in FIG. 4, that band 408 could be a suitable band for carbon dioxide detection, and it shows a maximum of total absorbance in the place 602 where the 10 nm-width band is located. FIG. 6b shows the relative water absorbance in the same 1598 nm-1616 nm band range, and it shows that, for the same 602 location, there is a relative minimum of water absorbance. FIG. 6c shows the product of the total (of all the gasses present) absolute and relative absorbance in the same 1598 nm-1616 nm band range. FIG. 6d shows the relative methane absorbance in the same 1598 nm-1616 nm band range, and it can be seen that methane absorbance is quite low in the location 602. FIG. 6e shows the relative absorbance of carbon dioxide in the same 1598 nm-1616 nm band range. From FIGS. 2b, 2c, 2d and 2e it can be seen that there is a maximum absorbance of only carbon dioxide in the position 602 where the 10 nm-width band is located. Therefore, if the band to detect carbon dioxide absorption is placed exactly in this position 602, carbon dioxide absorbs a lot considered alone, it is also the band where the carbon dioxide in absolute terms absorbs the most, and it is the band where, in general, the total absorbance is the highest. That is why this is a suitable position to place the carbon dioxide band. This is an example of how criteria such as absolute and/or relative total or partial absorbance of the target substance and potentially other substances, can be used to determine a suitable position for a band for detecting a certain target substance. In this example, in the selected position for the band, carbon dioxide absorbs a lot alone, it is also the band where it absorbs the most, alone and in total, it is the band where water and methane absorb the least or almost the least, and it is the band with the highest total absorbance.

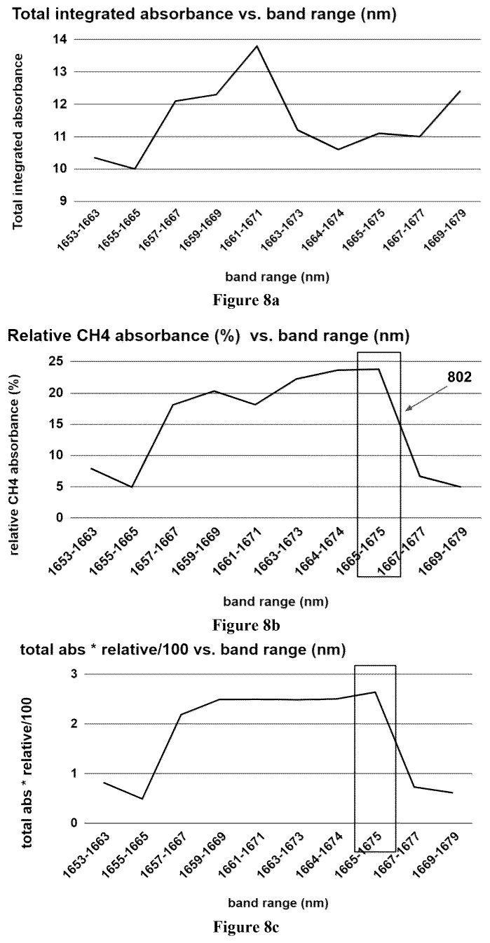

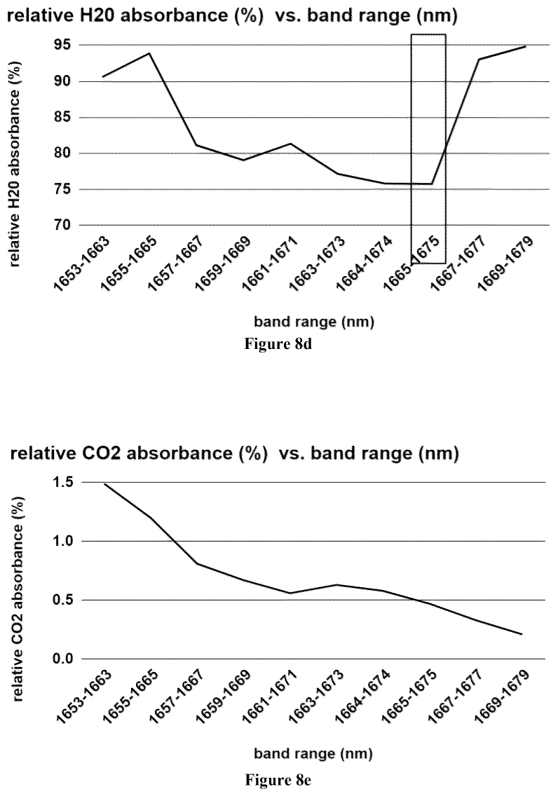

FIG. 7 shows the methane absorption band 702, based on the thin band 414 detected in FIG. 4 as suitable for methane detection. It is a band of 10 nm (as seen above, the determination of the width of a band is based on a tradeoff between collecting enough photons and providing enough spectral selectivity, considering the target substance and the spectral range where it is to be measured, and 10 nm may be considered as a suitable width). FIG. 8 shows examples of the criteria to select a suitable location for the methane absorption band 702. FIG. 8a shows the absorbance of all gasses present in the 1653 nm-1679 nm band range. FIG. 8a shows that, in the center, there is an absorption maximum. But in percentage, as seen in FIG. 8b, which shows the relative methane absorbance in the same 1653 nm-1679 nm band range, the methane band would absorb more in a position 802 more towards the right (towards the red spectrum). In the center, the methane absorption would be around 17%, but in the position 802 shown in FIG. 8b it would be around 25%. FIG. 8c, which shows the product of the total (of all the gasses present) absolute and relative absorbance in the same 1653 nm-1679 nm band range, even shows that the total absorbance is higher in that position 802 of the 10 nm-width band. FIG. 8d shows the relative absorbance of water in the same 1653 nm-1679 nm band range. It is interesting that, as seen in FIG. 8d, for the same position 802, the water absorbance is little, and FIG. 8e shows that the relative absorbance of the carbon dioxide in the same 1653 nm-1679 nm band range is in general very low. Hence, the position 802 provides a very good methane absorption, and a lower water and carbon dioxide absorption, which makes it a very pure band, and it does not correspond to the center of the methane absorption band 702 as shown in FIG. 7, but to a position shifted to one side. It can therefore be seen that it may be advantageous, in order to decide the position where a band is to be selected, to consider certain criteria that may include at least one of the total absolute and/or relative absorbance, the absolute and/or relative absorbance of the different substances detectable for different positions of the band, including the target substance and other substances, to determine how pure the band is. In this example, the position 802 of the 10 nm-width band is not a location where the total absorbance is the highest, but it is the location where methane absorbs the most, and where the water and carbon dioxide absorbance is low or the lowest.

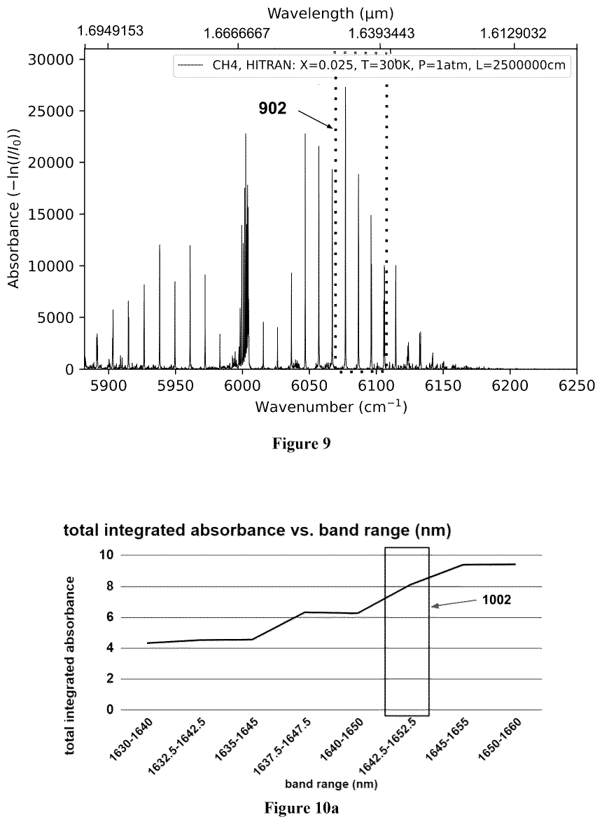

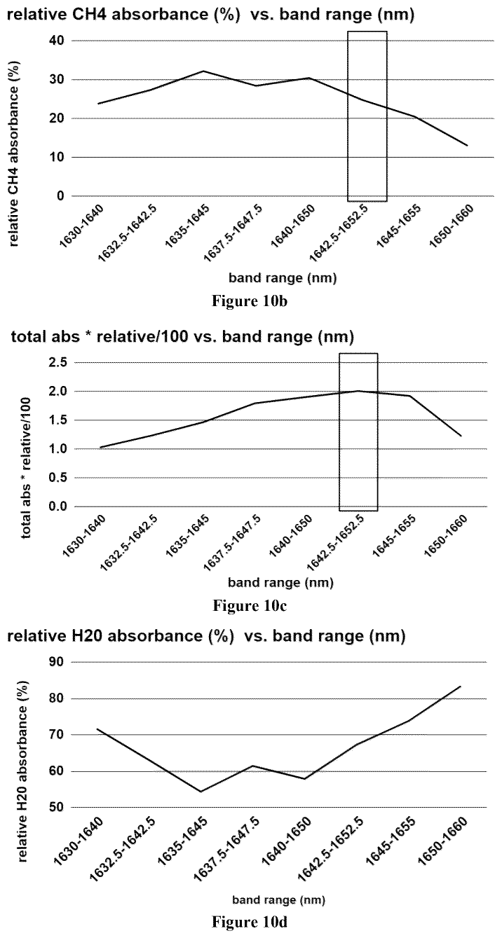

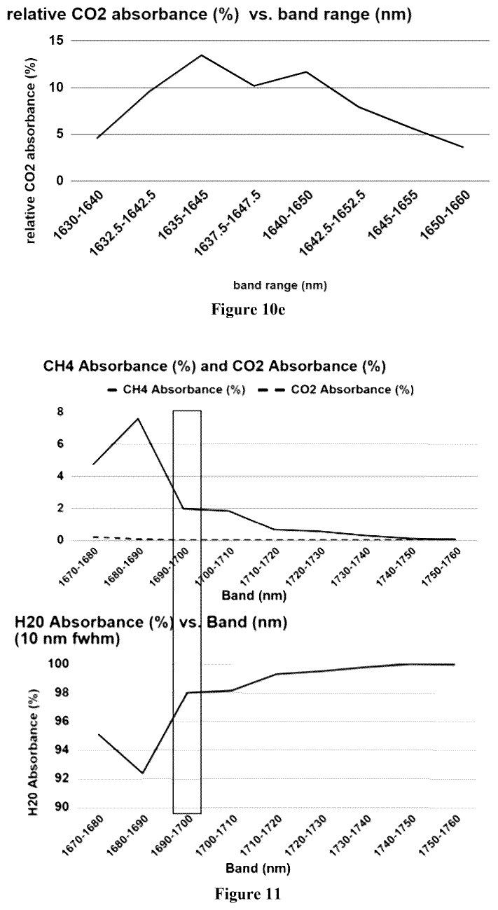

FIG. 9 also shows a methane absorption band, in this case methane absorption band 902, represented by a dashed line window and based on band 412 of FIG. 4 which was determined to be a candidate for methane detection. In this methane absorption band 902, many separate lines of absorption of methane are present, and it is a bit more difficult to decide in this area where to locate the band. FIG. 10 shows examples of the criteria to select a location for the methane absorption band 902, between 1630 nm and 1650 nm. For example, as seen in FIG. 10, at larger wavelengths there is more absorption as seen in FIG. 10a, which shows the absorbance of all gasses present in the band range of 1630 nm to 1650 nm. However, larger wavelengths have less relative methane absorption as seen in FIG. 10b, which shows the relative methane absorption in the same band range of 1630 nm to 1650 nm. In position 1002, however, there is an area where there is maximum total absorption, as seen in FIG. 10c, which shows the product of the total (of all the gasses present) absolute and relative absorbance in the same band range of 1630 nm to 1650 nm. Based on this information, and in view of the relative water absorbance, as seen in FIG. 10d, and the relative carbon dioxide absorbance, as seen in FIG. 10e, several band positions can be chosen, depending on the system or on the equipment, or on other specific criteria. In some embodiments, it might be decided to choose position 1002 which provides a maximum total absorbance, and therefore allows to increase the SNR of the detected signal, at the expense of losing some spectral selectivity by not choosing the more pure methane absorption area. In other embodiments, it might be decided to choose a position in which the relative methane absorbance is maximum based on FIG. 10b. In order to decide a suitable band for methane absorption, a balance of criteria needs to be found.

FIG. 11 shows a graphical representation of a suitable reference band or albedo band, which is to be located within the 1670 nm-1760 nm band range. This 1670 nm-1760 nm band range may be selected based at least in part on the band 402 shown in FIG. 4 as a potential candidate for a reference band. For this albedo band, what happens is that there is methane absorption, but there is also water absorption, there is no spectral area where there is no water. Hence, a location needs to be decided for the albedo band, where at least the methane absorption can be minimized. As can be seen in the upper graph of FIG. 11, the methane absorption (and the carbon dioxide absorption), if one moves from 1670 nm to 1760 nm, goes down. In the lower graph and in all the spectroscopic calculations of this document the atmospheric humidity has been set at 2.5%. It should be understood these specific values are for the purpose of exemplifying the concepts, without limiting the embodiments to specific values or amounts.

This can also been seen in Table 1 shown below:

| Band (nm) | CH4 | H20 | CO2 |
|---|---|---|---|
| (10 nm fwhm) | Absorbance (%) | Absorbance (%) | Absorbance (%) |
| 1670-1680 | 4.7 | 95.1 | 0.19 |
| 1680-1690 | 7.59 | 92.35 | 0.068 |
| 1690-1700 | 1.96 | 98 | 0.003 |
| 1700-1710 | 1.83 | 98.16 | 0.01 |
| 1710-1720 | 0.65 | 99.3 | 0.007 |
| 1720-1730 | 0.54 | 99.5 | 0.0004 |
| 1730-1740 | 0.28 | 99.78 | 4.5E−05 |
| 1740-1750 | 0.09 | 99.99 | 5.8E−06 |
| 1750-1760 | 0.05 | 99.95 | 0.003 |

In some instances, the bandwidths and/or the cutoff wavelengths of the bandwidths of the reference bands are selected based, at least in part, on the absorbance curves of water and at least one target substance (i.e. one or more substances to be detected). For example, taking into account the absorbance of CH₄, CO₂ and H₂O as a function of wavelengths shown in FIG. 11, within the range of 1670 nm to 1760 nm, the CH₄ absorbance values start decreasing compared to the H₂O absorbance values, which start increasing, hence the bandwidth cutoff wavelength of at least one reference band may be selected within the range of 1670 nm to 1760 nm, when the CH₄ absorbance values start decreasing, or when the CH₄ absorbance values decrease to below a certain absorbance threshold, which is a threshold enough to consider that a certain substance is present, or has a relevant presence. In some instances, the bandwidth of at least one reference band of a band-pass filter may be selected as centered at around 1690 nm. Preferably, the bandwidth of at least one reference band is located at higher wavelengths in the spectrum of electromagnetic radiation, where the absorbance of CH₄ is low and the H₂O absorbance is high. Hence, the reference band located at higher wavelengths where the absorbance of CH₄ is low, depends on the water content on the Earth. However, since it is expected that the variation of water content on the Earth is relatively constant, if there is a gas (CH₄) leak, the absorbance of CH₄ should abruptly increase while the water content on the Earth (for example along a 100 meters distance) would not increase significantly. Hence, an approach for selecting a reference band (also referred to as albedo) includes not necessarily selecting a pure band where there is no absorption of any substance, but where there is absorption of other substance(s) than the target substance(s), such that the absorbance of the other substance(s) in the selected spectral range is expected to vary slowly and in a small amount. In the present example, such a band may be seen as a relatively pure band (with respect to methane) given that there is low methane absorption.

In some instances, the bandwidth of the reference band may be further selected, based at least in part on a property or characteristic of the detector, such as the quantum efficiency of the detector. For example, the bandwidth of the reference band may be from about 1690 nm to about 1700 nm.

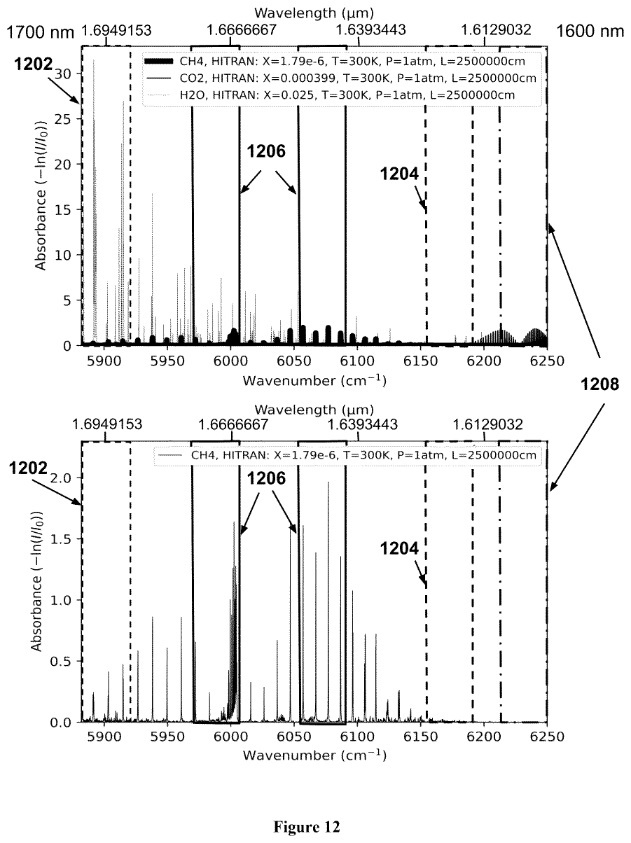

In some embodiments, the multispectral filter may have at least two reference bands. FIG. 12 shows the reference band 1202 centered at around 1695 nm, corresponding to the reference band discussed above with respect to FIG. 11, and the reference band 1204 centered at around 1620 nm. This band 1204 may be selected based at least in part on the band 406 shown in FIG. 4 as a potential candidate for a reference band. FIG. 12 shows the absorbance as a function of wavelength for (A) H₂O in the upper graph and (B) CH₄ in the lower graph, and it can be seen that the reference band 1202 is a relatively pure CH₄ band since there is low methane absorption but high water absorption, and the reference band 1204 is a pure band, as it has little or no CH₄ or H₂O absorption. FIG. 12 also shows bands 1206, which are the CH₄ absorption bands, and band 1208, which is the CO₂ absorption band.

With the selection of at least two reference bands, an interpolation function may be done having the measurements at the reference bands as interpolation points. The interpolation function created with the measurements of at least two reference bands may provide the relative variation, which is not considered in prior art approaches. It is possible to use pure bands or relatively pure bands because the bands are sufficiently narrow. In instances in which there is at least one reference band, the interpolation function may be built based on the measurements at the at least one reference band, or it may even correspond to a single (interpolation) point/value. The interpolation function can be an interpolation curve, and can be obtained with linear interpolation, polynomial interpolation or any other type of suitable interpolation. The type of interpolation function may be determined based on characteristics such as the spectral structure of the target substance(s), and hence the position and/or width of the target band(s). In the example shown above, there are two methane absorption bands located spectrally close to each other, at one side of both there is a spectrally pure band, and at the other side of both there is a band with 96%-98% water absorption, and therefore a simple linear interpolation may be suitable to determine the tendency of the albedo. However, if the methane absorption bands are in a different position, or are more separated, it might be advantageous to have at least one additional reference band in order to have at least one additional interpolation point. What is important is that the interpolation points, and hence the reference bands, are selected so that the interpolation can provide a reliable representation of the variation of the albedo throughout the spectral range of interest. The number and position of the interpolation points may be determined based on the number of target bands, the spectral distance among them, and the frequency of variation of the albedo in the desired spectral range.

Prior art approaches select one band where there is a high methane absorption and one band next to it where there is low methane absorption and obtain the ratio between measurements, to make a kind of normalization in order to detect methane removing the background. In contrast, in embodiments of present disclosure, the approach is to build a type of baseline with interpolation points obtained from two or more reference bands with the characteristics described throughout this description. The interpolation curve built from the interpolation points obtained based on the reference bands allows to obtain accurate target substance measurements in order to detect and eventually quantify the amount of target substances, reducing or eliminating the incidence or effect of the albedo. This is particularly useful in systems with limited resources which may need to increase the SNR of signals due to the small and economical components of the detection systems.

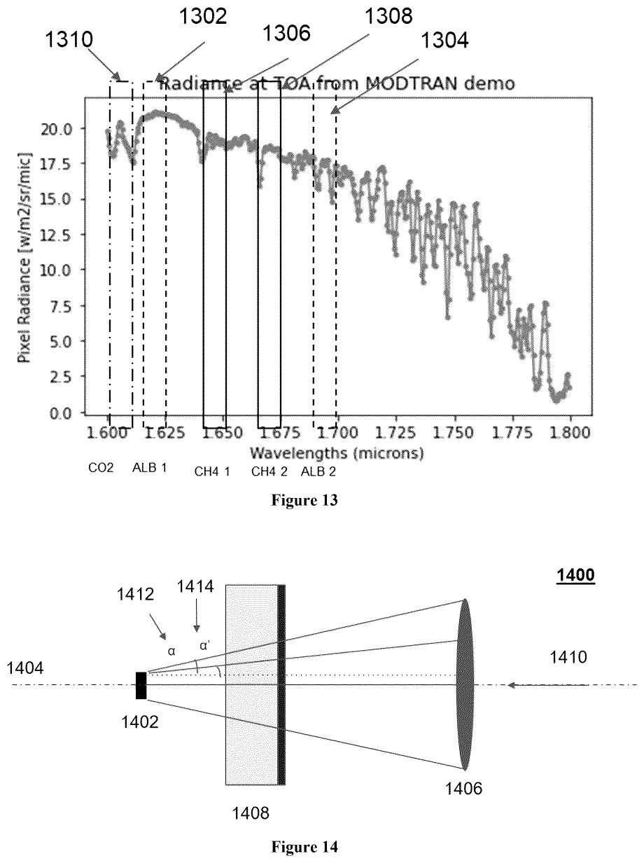

FIG. 13 illustrates the radiance on Top Of Atmosphere (TOA) between 1600 nm to 1800 nm, which is what a satellite or spacecraft, such as a low Earth orbit satellite, may measure. In other words, the signal that the satellite or spacecraft measures to detect the presence of substances is the radiance on Top of Atmosphere. Generally, the satellite actually measures a spectrum wherein the components of low frequency profile (low frequency variation, referring to the Fourier transform of the spectrum for which the albedo has typically a smooth curve) are used to retrieve the albedo, while the high frequency ones are used for the substance concentration retrieval. The albedo depends on the reflectance of the Earth, the scattering effects of the atmosphere, aerosol particles, and other factors present between the Earth and the satellite that influence the measurements. For example, for methane detection, the relevant measurements are the negative peaks (minima) on the spectrum that are within the region of methane absorption; and likewise for other gasses absorption, such as carbon dioxide negative peaks for carbon dioxide detection. The bands 1302, 1304, 1306, 1308 and 1310 are the bands of the multispectral band-pass filter according to some embodiments of the present disclosure. For instance, the band 1302 between about 1615 nm and 1625 nm corresponds to a first measurement of albedo (reference band, pure albedo), and corresponds at least in part to band 1204 in FIG. 12, which may be determined based a least in part on band 406 of FIG. 4. The band 1304 between about 1690 nm and 1700 nm corresponds to a second measurement of albedo (reference band, complex albedo), and corresponds at least in part to band 1202 in FIG. 12, also based at least in part on the band shown in FIG. 11, and which may be determined based a least in part on band 402 of FIG. 4. The band 1306 between about 1642-1652 nm corresponds to a first measurement of methane absorbance, and corresponds at least in part to band 902 of FIG. 9 and band 1002 of FIG. 10, which may be determined based at least in part on band 404, more particularly on peaks 412 of FIG. 4. The band 1308 between about 1665 nm and 1675 nm corresponds to a second measurement of methane absorbance, and corresponds at least in part to band 702 of FIG. 7 and band 802 of FIG. 8 (indeed the actual location of the band may be adapted to the criteria considered, as explained with relation to FIG. 8), which may be determined based at least in part on band 404, more particularly on peaks 414 of FIG. 4. Lastly, the band 1310 between about 1600 nm and 1610 nm corresponds to a measurement of carbon dioxide absorbance, and it corresponds at least in part to band 504 of FIG. 5, which may be determined based at least in part on band 408 of FIG. 4.

In some instances, the filter, the detector or both may include at least one coating to prevent crosstalk between bands. For example, a focal plane array may be coated in order to detect photons including the desired bands (e.g. 1600 nm to 1700 nm) and reduce or eliminate the detection of photons outside the bandwidths of the desired bands (e.g. less than 1600 nm). Crosstalk between bands refers to the phenomenon that occurs when photons falling on one pixel are “falsely” sensed by other pixels around it.

Embodiments include methods comprising obtaining measurements of at least two reference bands. In certain embodiments, one band is spectrally pure, in other words, there is almost (below a pure band threshold) no gas or substance absorbing at that range of wavelengths (e.g. band 1302) and another band is spectrally relatively pure, in other words, there is high (equal to or above an absorbance threshold) absorption of at least one substance different than the target substance based at least in part on a priori information about substances (likely) present in that range of wavelengths, it can be determined that said band can be used as reference band, for example, a band where there is high water absorption (e.g. band 1304 with a water absorption of around 98%). In such a band, there may also be absorption of the target substance, however it may be determined that the relative absorption of the target substance with respect to the relative absorption of the other at least one substance is low enough to consider that the frequency variation of the albedo can be obtained from the absorption of the other at least one substance. In some embodiments, the pure band threshold and the absorbance threshold may be identical. The reference bands or albedo bands may also be understood as disambiguation bands, because they may be used to disambiguate the absorbance of the target substance from other substances different from the target substance, and which are expected to be present in the target location and in the target spectral area (a priori information). If for a certain multispectral image, at around 1600 nm, it is detected that there is information being measured, it provides an indication that there is something absorbing. However, it is necessary to determine which substance(s) is/are absorbing from among the substances that may absorb around the same spectral area. For example, if detected that there is absorption in a spectral area where it is known that methane and water absorb, a reference band may be selected, in such a place where water absorption is high and methane absorption is low.

FIG. 14 illustrates a side view of an example substance detection device 1400 according to embodiments of the present disclosure, having a focal plane array 1402 aligned with respect to an optical axis 1404 of a lens 1406. The focal plane array 1402 includes a plurality of pixel sensors arranged in a planar area that coincides with the optical axis 1404 of the lens 1406. A bandpass filter 1408, such as a multispectral optical filter, is disposed in the optical path of the focal plane array 1402 and may be used to filter the incoming light 1410. Light enters the lens 1406 (from the right side of the FIG. 14) and passes through the bandpass filter 1408, reaching the planar surface of focal plane array 1402. The substance detection device 1400, which may be part of, or correspond to, a substance detection system, may be part of an aerial or satellite system, such as part of a satellite. The substance detection device 1400 may likewise be part of a ground-based system. In some embodiments, the substance detection device 1400 may be part of an imaging system on an aerial, satellite or ground-based system. Additionally or alternatively, the substance detection device 1400 may be placed on a platform, which in some instances, may not be designed to move during substance detection, while in other implementations, the platform is in motion during substance detection.

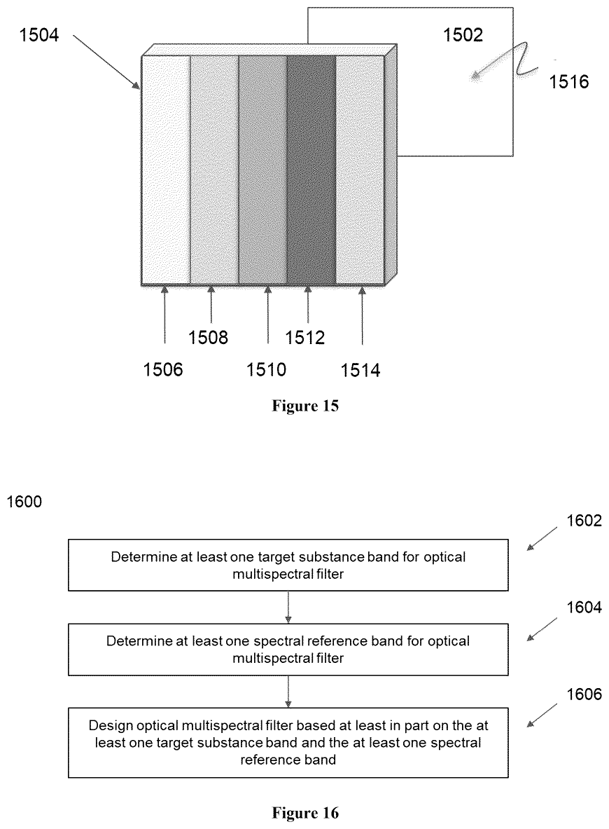

The substance detection device 1400 may be part of a primary payload of a small system having limited resources, such as a micro or nano-satellite system where there is limited power or space available and the weight of the whole system needs to be kept to a minimum, or may be part of a secondary payload of that system, or even as a hosted payload. In some instances, the bandpass filter 1408 may be a dichroic filter, and its operation may be related with an angle of incidence of light passing through the filter, generally designed for operating with light entering perpendicularly to the surface of the filter. Hence, before passing through the filter and reaching the focal plane array, the light should be collimated, since the width of the incoming light 1410 reaching the focal plane array 1402 depends on the “F” number of the lens 1406, which limits the number of photons collected by the pixel sensors of the focal plane array 1402. However, in a system having limited resources, or which should reduce or limit its weight, it may be undesirable to employ or add additional resources for collimating the light reaching the focal plane array 1402. The angle 1412 and 1414 of light reaching the focal plane array 1402 after passing through the bandpass filter 1408 depends on the F number of the lens, which in general does not change shape of the bands or the light transmission but produces a spectral shift of the bands (wavelengths of light) towards the blue end of the electromagnetic spectrum. Consequently, the central wavelengths of the bands of the bandpass filter 1408 may be chosen based at least in part on the spectral shift of the light passing through the lens and the bandpass filter. The spectral shift is related to the characteristics of the lens, generally the F number of the lens. In some instances, the band's central wavelength blue shift of an optical filter that is a dichroic filter may be calculated according to the light angle of incidence, due to the F number of the lens or a COTS directing, collecting and/or focusing element. FIG. 15 illustrates an example of a detector 1502, and a multispectral optical filter 1504. The multispectral optical filter 1504 may be an interference filter made of glass, or other suitable material, wherein the sections or bands may be etched layers of a semiconductor material deposited on the glass, however other techniques known in the art may be also used. The position of the sections or bands of the multispectral optical filter may be selected in order to minimize the crosstalk between bands. For instance, the sections or bands may be arranged so that one band (e.g. bands 1506, 1510 and 1514) has a higher refractive index than the band next to it (e.g. bands 1508, 1512).

The design of the sections or bands, including a determination of the amount of sections or bands, the spectral location where they are to be located, and their bandwidth, can be performed as explained elsewhere within this Detailed Description. The filter 1504 may comprise one or more filter sections, wherein at least one of the one or more filter sections is a target substance filter section for detection of at least one target substance and at least another one of the one or more filter sections is a spectral reference filter section.

As described elsewhere within this Detailed Description, the at least one spectral reference filter section and the at least one target substance filter section may be determined based at least in part on at least one of: a spectral proximity between at least one spectral reference band and at least one target substance band, a spectral proximity between several target reference bands, a correlation between at least one spectral reference band and at least one target substance band, a correlation between several target reference bands, a maximum total absorbance of at least one substance present in a spectral area of interest, a minimum total absorbance of at least one substance present in the spectral area of interest, a maximum relative absorbance of at least one substance present in the spectral area of interest, a minimum relative absorbance of at least one substance present in the spectral area of interest, a maximum between the total and relative absorbance, or a minimum product between the total and relative absorbance.

For example, the one or more target substance filter sections may be determined to comprise one first target substance band for carbon dioxide detection, and preferably one second target substance band for methane detection and one third target substance band for methane detection, wherein preferably the first target substance band for carbon dioxide detection is centered at around 1600 nm, the second target substance band for methane detection is centered at around 1647 nm and the third target substance band for methane detection is centered at around 1670 nm.

For example, the one or more spectral reference filter sections may be determined to comprise at least one spectral reference filter section centered at around 1620 nanometers and at least one spectral reference filter section centered at around 1695 nanometers.

The detector 1502 may further comprise a coating 1516 on its back surface. The front surface is understood as the surface receiving the incoming light, and the back surface refers to the surface opposite to the front surface. In some instances, the multispectral optical filter 1504 may have a size bigger than the detector 1502, hence, in order to minimize or reduce any undesired light from reaching the front surface of the detector 1502, the back surface multispectral optical filter 1504 may further comprise a coating to reduce or minimize the crosstalk between detection signals. Apart from a multispectral optical filter layout wherein the filter sections are arranged as discrete filter bands in which each filter band allows the transmission of a wavelength range generally defined by a central wavelength, it should be understood that other layouts or arrangements of filter sections in the optical filter are also envisaged in the embodiments of present disclosure, such as filter arrays or mosaics, or any combination of discrete filter bands and filter arrays or mosaics.

FIG. 16 shows a method 1600 to design a multispectral filter according to some embodiments of the present disclosure. A method according to some embodiments comprises, in step 1602, determining at least one target substance band for the optical multispectral filter. In step 1604, the method comprises determining at least one spectral reference band for an optical multispectral system to be used in a remote substance detection system. In step 1606, the method comprises designing the optical multispectral filter based at least in part on the at least one target substance band and the at least one spectral reference band. It is to be understood that steps 1602 and 1604 may be performed in a different order. According to certain embodiments, the optical multispectral filter has at least four spectral bands. The method may further comprise, to design the optical multispectral filter, performing a comparison or determining a difference between the absorbance values within a first spectral band and a second spectral band, to obtain information from at least one target substance, wherein the first spectral band of the at least four spectral bands is sensitive to the at least one target substance (e.g. methane), and the second spectral band of the at least four spectral bands is insensitive to the at least one target substance.

The method according to some embodiments may further comprise, to design the optical multispectral filter, obtaining an interpolation curve with values of a third spectral band (a first reference band which may correspond to the at least one spectral reference band) and a fourth spectral band (a second reference band). The determination of an interpolation curve allows to homogenize the radiance values of the pixels, in order to obtain uniform radiance values for the bands. This is useful, since the radiance from the surface on the Earth collected by each pixel may not be similar or of the same order of magnitude for all the pixels.

Hence, the method according to some embodiments comprises, on one hand, the determination of the target substance, in some embodiments performed pixel by pixel, by comparing or contrasting the radiance on Top of Atmosphere values of predetermined spectral bands (e.g. the first and second spectral bands) and, on the other hand, the uniformization of the image using the reference bands (e.g. the third and fourth spectral bands). In some instances, the reference bands are located on each side of the spectral bands or target substance bands (e.g. the first and second spectral bands) for detecting the at least one target substance.

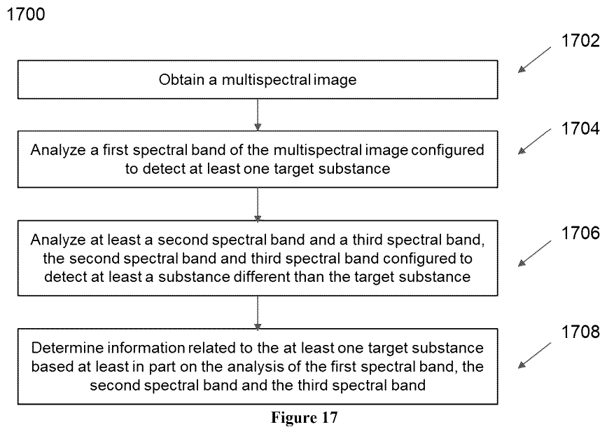

FIG. 17 shows a method 1700 for data analysis according to embodiments of the present disclosure. The method is based at least in part on an architecture of the system for remote substance detection described throughout this Detailed Description, and it is to maximize signal to noise ratio, SNR, for a target substance detection. In step 1702, the method comprises obtaining a multispectral image. This multispectral image may be obtained in different ways, such as by capturing the multispectral image with a sensor on board the system for substance detection, or by retrieving the multispectral image from a memory or a database where it may be stored, or by receiving the multispectral image from another device or system. In step 1704, the method comprises analyzing a first spectral band (target substance band) of the multispectral image, wherein the first spectral band is configured to detect at least one target substance, in other words, where the at least one target substance is detectable by the first spectral band. In step 1706, the method comprises analyzing at least a second spectral band (spectral reference band) and a third spectral band (spectral reference band), wherein at least the second spectral band and the third spectral band are configured to detect at least a substance different than the target substance, in other words, wherein the target substance is not detectable, or is detectable below a certain pure band threshold, or is detectable but other substance(s) are detectable with higher absorbance and the detection of the target substance can be considered negligible. In step 1708, the method comprises determining information related to the at least one target substance based at least in part on the analysis of the first spectral band and on the analysis of the second spectral and the third spectral band.

It is to be appreciated that embodiments of the methods and apparatuses discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiment.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

In the descriptions above and in the claims, phrases such as “at least one of or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B:”, “at least one of A or B:”, “one or more of A and B:”, and “A and/or B’ are each intended to mean “A alone, B alone, or A and B together.” A similar interpretation is also intended for lists including three or more items. For example, the phrases “at least one of A, B, and C:”, “one or more of A, B, and C:” and “A, B, and/or C” are each intended to mean “A alone, B alone, C alone, A and B together, A and C together, B and C together, or A and B and C together.” In addition, use of the term “based on” above and in the claims is intended to mean “based at least in part on”, such that an unrecited feature or element is also permissible.

### CONCLUSION

The subject matter described herein can be embodied in systems, apparatus, methods, and/or articles depending on the desired configuration. Although the disclosure uses language that is specific to structural features and/or methodological acts, the invention is not limited to the specific features or acts described. Rather, the specific features and acts are disclosed as illustrative forms of implementing the subject matter described herein. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1-29. (canceled)

30. A remote substance detection system, the system comprising:
at least one light directing element;
at least one optical multispectral filter; and
at least one detector;
wherein the optical multispectral filter is configured to have at least one target substance band for detection of at least one target substance and at least one spectral reference band.

31. The system according to claim 30, wherein the at least one spectral reference band and the at least one target substance band are determined based at least in part on at least one of:
a spectral proximity between the at least one spectral reference band and the at least one target substance band; a spectral proximity between several target reference bands; a correlation between the at least one spectral reference band and the at least one target substance band; a correlation between several target reference bands; a maximum total absorbance of at least one substance present in a spectral area of interest; a minimum total absorbance of at least one substance present in the spectral area of interest; a maximum relative absorbance of at least one substance present in the spectral area of interest; a minimum relative absorbance of at least one substance present in the spectral area of interest; a maximum between the total and relative absorbance; or a minimum product between the total and relative absorbance.

32. The system according to claim 30, wherein the at least one spectral reference band is selected to be located in spectral areas where an absorbance of the at least one target substance is lower than an absorbance threshold or lower than a pure band threshold, and/or in spectral areas where a relative absorbance of the at least one target substance with respect to other substance or substances is low enough that a change in absorption of the other substance or substances does not depend on a change in the at least one target substance.

33. The system according to claim 30, wherein an amount, position in the spectrum and/or bandwidth of the at least one spectral reference band is based at least in part on a number of target substance bands, a spectral distance between the target substance bands, and a frequency of variation of albedo in a spectral range of interest.

34. The system according to claim 30, wherein the optical multispectral filter is configured to have at least two spectral reference bands; and wherein an interpolation curve is obtained using measurements at the at least two spectral reference bands as interpolation points, in order to obtain a relative variation, wherein preferably the measurements correspond to values of a radiance on top of atmosphere.

35. The system according to claim 34, wherein a first reference band is centered at around 1620 nanometers and a second reference band is centered at around 1695 nanometers.

36. The system according to claim 30, further comprising a coating layer.

37. The system according to claim 30, wherein the at least one multispectral optical filter comprises at least one of:
one first target substance band for methane detection, preferably centered at around 1647 nm; one second target substance band for methane detection, preferably centered at around 1670 nm; or one third target substance band for carbon dioxide detection, preferably centered at around 1600 nm.

38. An optical filter comprising:
one or more filter sections, wherein at least one of the one or more filter sections is a target substance filter section for detection of at least one target substance and at least another one of the one or more filter sections is a spectral reference filter section;
wherein the optical filter is configured to be disposed in an optical path of a lens and focal plane array, and is configured to filter light that passes through the lens to reach a planar surface of the focal plane array.

39. The optical filter according to claim 38, wherein the at least one spectral reference filter section and the at least one target substance filter section are determined based at least in part on at least one of: a spectral proximity between at least one spectral reference band and at least one target substance band; a spectral proximity between several target reference bands; a correlation between the at least one spectral reference band and the at least one target substance band; a correlation between several target reference bands; a maximum total absorbance of at least one substance present in a spectral area of interest; a minimum total absorbance of at least one substance present in the spectral area of interest; a maximum relative absorbance of at least one substance present in the spectral area of interest; a minimum relative absorbance of at least one substance present in the spectral area of interest; a maximum between the total and relative absorbance; or a minimum product between the total and relative absorbance.

40. The optical filter according to claim 38, wherein the target substance filter section is at least one of:
a first target substance band for methane detection, preferably centered at around 1647 nm;
a second target substance band for methane detection, preferably centered at around 1670 nm; or
a third target substance band for carbon dioxide detection, preferably centered at around 1600 nm.

41. The optical filter according to claim 38, wherein the one or more filter sections comprise one first target substance band for carbon dioxide detection, and preferably one second target substance band for methane detection and one third target substance band for methane detection, wherein preferably the first target substance band for carbon dioxide detection is centered at around 1600 nm, the second target substance band for methane detection is centered at around 1647 nm and the third target substance band for methane detection is centered at around 1670 nm.

42. The optical filter according to claim 38, wherein at least one spectral reference filter section is centered at around 1620 nanometers and at least one spectral reference filter section is centered at around 1695 nanometers.

43. The optical filter according to claim 38, wherein the one or more filter sections are arranged as discrete filter bands, filter arrays or mosaics, or any combination of these.

44. A computer-implemented method of designing an optical filter for remote substance detection, comprising the steps of:
determining at least one target substance band for an optical multispectral filter to be used in a remote substance detection system;
determining at least one spectral reference band for the optical multispectral filter; and
designing the optical multispectral filter based at least in part on the at least one target substance band and the at least one spectral reference band.

45. The method according to claim 44, wherein determining the at least one target substance band is based at least in part on at least one of: a spectral proximity between the at least one spectral reference band and the at least one target substance band; a spectral proximity between several target reference bands; a correlation between the at least one spectral reference band and the at least one target substance band, a correlation between several target reference bands; a maximum total absorbance of at least one substance present in a spectral area of interest; a minimum total absorbance of at least one substance present in the spectral area of interest; a maximum relative absorbance of at least one substance present in the spectral area of interest; a minimum relative absorbance of at least one substance present in the spectral area of interest; a maximum between the total and relative absorbance; or a minimum product between the total and relative absorbance.

46. The method according to claim 44, wherein determining the at least one spectral reference band comprises determining at least two spectral reference bands, wherein preferably determining the at least two spectral reference bands comprises selecting the at least two spectral reference bands to be located in spectral areas where an absorbance of at least one target substance is lower than an absorbance threshold and/or in spectral areas where a relative absorbance of the at least one target substance with respect to other substance or substances is low enough that a change in absorption of the other substance or substances does not depend on a change in the at least one target substance.

47. The method according to claim 46, further comprising obtaining an interpolation curve using measurements at the at least two spectral reference bands as interpolation points, wherein preferably the measurements correspond to values of a radiance on top of atmosphere.

48. The method according to claim 44, wherein the method preferably comprises determining a number and position of the spectral reference bands based on a number of target substance bands, a spectral distance between the target substance bands, and a frequency of variation of albedo in a desired spectral range.

49. A computer-implemented method for data analysis, based at least in part on an architecture of the remote substance detection system of claim 30, to maximize signal to noise ratio, SNR, for a target substance detection, the analysis comprising:
obtaining a multispectral image;
analyzing a first target substance band of the multispectral image, wherein the first target substance band is configured to detect the at least one target substance;
analyzing at least a second spectral band and a third spectral band, wherein at least the second spectral band and the third spectral band are configured to detect at least a substance different than the at least one target substance; and
determining information related to the at least one target substance based at least in part on the analysis of the first target substance band and on the analysis of the second spectral and the third spectral band.
